(19) United States
(12) Patent Application Publication
(10) Pub. No.: US 2005/0215994 A1
(43) Pub. Date: Sep. 29, 2005

(54) Tunneling device

(76) Inventor: Stephen Solomon, Baltimore, MD (US)

Correspondence Address: FRISHAUF, HOLTZ, GOODMAN & CHICK, PC, 220 5TH AVE FL 16, NEW YORK, NY, 10001-7708, US

(21) Appl. No.: 10/922,274

(22) Filed: Aug. 19, 2004

Related U.S. Application Data
(60) Provisional application No. 60/496,812, filed on Aug. 21, 2003.
(60) Provisional application No. 60/505,081, filed on Sep. 22, 2003.

(51) Int. Cl. A61B 18/14

(52) U.S. Cl. 606/45; 606/48; 606/49; 606/50

(57) ABSTRACT

A tunneling device is provided which includes a substantially rigid rod, an energy emitting part at a distal end of the rod, a catheter holding part at a proximal end of the rod, an energy source connected to the energy emitting part, and a control section for activating the energy source to transmit energy to the energy emitting part, so as to cause the energy emitting part to emit cutting and/or cauterizing energy. The substantially rigid rod is adapted to be guided through body tissue while the energy source is active so as to facilitate movement and control of the guided rod.

### CROSS-REFERENCE TO RELATED APPLICATIONS

This application is based upon and claims the priority of U.S. Provisional Applications Ser. No. 60/496,812 filed Aug. 21, 2003 and Ser. No. 60/505,081 filed Sep. 22, 2003, the entire contents of both of which are incorporated herein by reference.

### FIELD OF THE INVENTION

The invention relates to a tunneling device for creating a path for a subcutaneous catheter.

The invention further relates to an apparatus for monitoring movement of a tunneling device along a subcutaneous path.

### BACKGROUND OF THE INVENTION

Several surgical/interventional procedures require tunneling through soft-tissue in order to create a path into which a catheter or graft may be inserted. One such procedure is the insertion of a ventriculo-peritoneal catheter, which allows excess fluid in the brain to be drained into the abdomen. In order to place the catheter, a subcutaneous tunnel is created through the soft tissue of the body from the head to the abdomen. Another such procedure is the insertion of a tunneled central venous catheter which enters a body just over the chest and travels subcutaneously to the internal jugular vein. Moreover, tunneling devices may be used to create paths for vascular grafting.

FIG. 1 shows a schematic view of a typical tunneling device. Currently, devices for creating a tunnel for the insertion of a catheter comprise only a rigid rod 1, a blunt distal end 4 of the rod 1 which is pushed through soft tissue, and a catheter 3 attached to a proximal end 2 of the rod 1. The device must be pushed through soft tissue to create a pathway for the catheter. However, it is difficult to push the rod through some types of tissue, and different degrees of force are necessary for different types of tissue. Indeed, the force required to push the device can be so large that the rod may move uncontrollably once tissue gives way under the force. In addition, tunneling through soft tissue sometimes causes bleeding which cannot be halted by the tunneling devices which are currently known.

Still further, when using a tunneling device, it is sometimes difficult for a surgeon to determine a location of the distal end of the rod within the body, forcing the surgeon to estimate the location of the distal end of the rod. This practice can be dangerous, especially traveling near lung and abdominal tissue, for example.

### OBJECT OF THE INVENTION

It is an object of the present invention to provide a device for tunneling through soft tissue which does not encounter the above-described problems of uncontrolled movement and bleeding.

It is an additional object of the present invention to provide an apparatus which can monitor the location of the distal end of the rod by monitoring the type of tissue which surrounds the rod.

### SUMMARY OF THE INVENTION

To achieve the object of the invention, a tunneling device is provided which comprises a substantially rigid rod, an energy emitting part at a distal end of the rod, a catheter holding part at a proximal end of the rod, an energy source connected to the energy emitting part, and a control section for activating the energy source to transmit energy to the energy emitting part, so as to cause the energy emitting part to emit cutting and/or cauterizing energy, wherein the substantially rigid rod is adapted to be guided through body tissue while the energy source is active so as to facilitate movement and control of the guided rod.

The energy emitting part may comprise an electrode cap which is monopolar or bipolar, and the energy source may comprise a Radio Frequency (RF) generator.

Alternatively energy source may comprise a battery housed in a casing that is directly connected to the substantially rigid rod.

Alternatively, the energy emitting part may comprise one of a laser tip and an ultrasonic tip and the energy source may comprise a laser power source or ultrasound generator respectively.

Alternatively, the energy emitting part may comprise a mechanical cutting tool.

The energy source may be connected to the energy emitting part by an insulated wire.

The control section may comprise a foot pedal, trigger, or other control mechanism.

The control section may further comprise a timing section for activating the energy source for a only a predetermined period of time.

The tunneling device may further comprise a tube attached to the substantially rigid rod and a syringe connected to a proximal end of the tube, wherein the syringe and tube are adapted to deliver antibiotic or anesthetic medications at the distal end of the substantially rigid rod.

In an alternative means of achieving the object of the invention, a tunneling device is provided which comprises a metallic rod insulated except at a distal end thereof, an energy source connected to the metallic rod, and a control section for activating the energy source to transmit energy to the energy emitting part, so as to cause the energy emitting part to emit cutting and/or cauterizing energy, wherein the metallic rod is adapted to be guided through body tissue while the energy source is active so as to facilitate movement and control of the guided rod.

The energy source may comprise a Radio Frequency (RF) generator and may be connected to the metallic rod by an insulated wire.

The control section may comprise a foot pedal, trigger, or other control mechanism.

The control section may further comprise a timing section for activating the energy source for a only a predetermined period of time.

The tunneling device may further comprise a tube attached to the insulated metallic rod and a syringe connected to a proximal end of the tube, wherein the syringe and tube are adapted to deliver antibiotic or antiseptic medications at the distal end of the metallic rod.

In another alternative means of achieving the object of the invention, a tunneling device is provided which comprises a pair of metallic rods insulated on an exterior surface except at a distal end thereof and separated by insulation except at a proximal and a distal connection point, an energy source connected to the metallic rods, and a control section for activating the energy source to transmit energy to the energy emitting part, so as to cause the energy emitting part to emit cutting and/or cauterizing energy, wherein the metallic rod is adapted to be guided through body tissue while the energy source is active so as to facilitate movement and control of the guided rod.

To achieve the additional object of the invention, a tissue impedance measuring apparatus is provided which comprises an impedance measuring section for measuring tissue impedance at the location of the distal end of the rod, and a display section for displaying impedance data measured by the impedance measuring section.

The apparatus may further comprise an alarm generating section for generating an alarm when a predetermined impedance parameter is exceeded.

### BRIEF DESCRIPTION OF THE DRAWINGS

FIG. 1 is a schematic view of a conventional tunneling device.

FIG. 2 is a schematic view of the present invention.

FIG. 3 is a schematic view of a second embodiment of the present invention.

FIG. 4 is a schematic view of a third embodiment of the present invention;

FIG. 5 is a schematic view of a fourth embodiment of the present invention; and

FIG. 6 is a schematic view of a fifth embodiment of the present invention.

### DETAILED DESCRIPTION

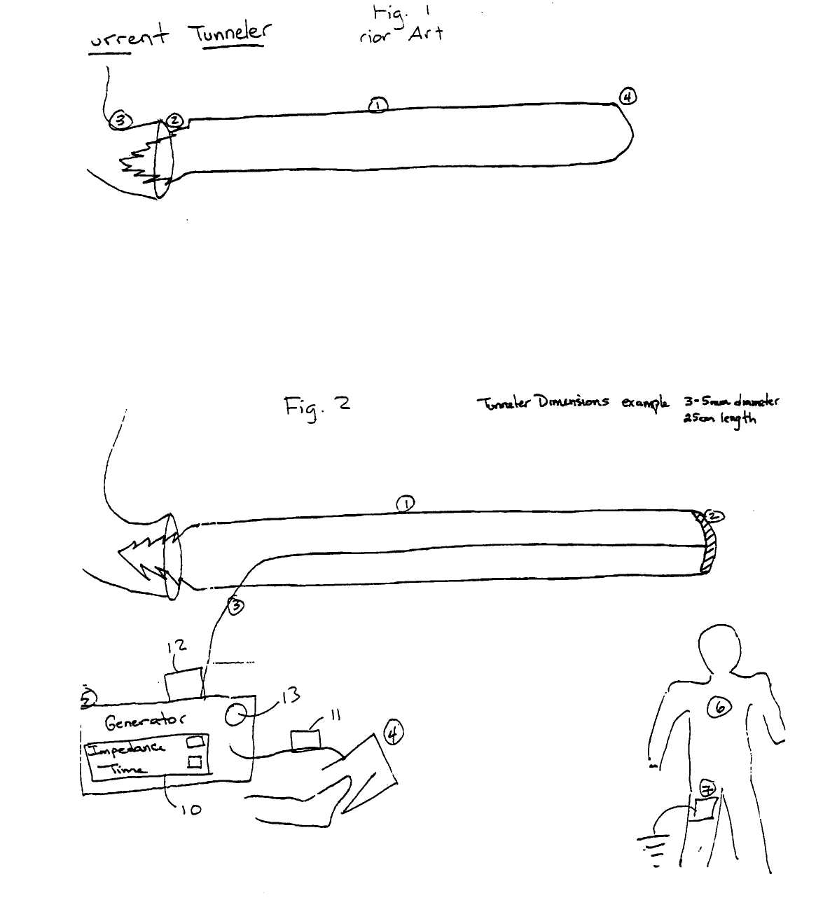

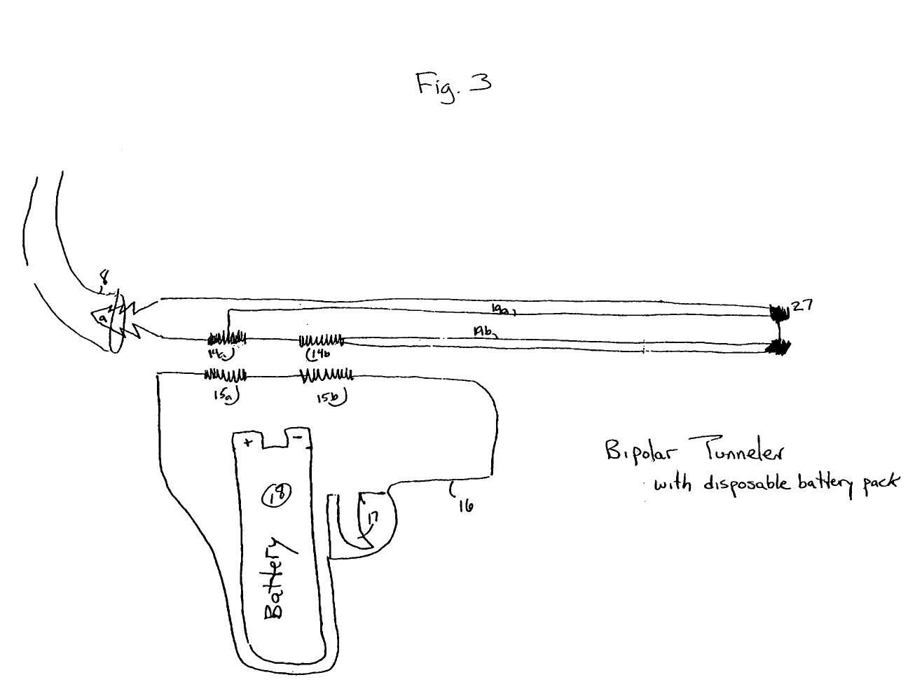

FIG. 2 shows a schematic view of a first embodiment of the present invention. A substantially rigid rod 1 has an electrode cap 2 attached to a first end of the rod 1 and a catheter 8 is attached to a catheter holding part 9 of a second end of the rod 1. A Radio Frequency (RF) generator 5 is attached to the electrode cap 2 by an insulated wire 3 which enters the rod 1 near the catheter holding part 9, runs through a center portion of the rod 1, and attaches to a side of the electrode cap 2 which is connected to the rod 1. A foot pedal 4 is connected to the RF generator 5.

The device is utilized by first placing a grounding pad 7 on a patient 6. The rod 1, to which the catheter 8 is attached, is inserted into an entry point incision and the RF generator 5 is activated by depressing the foot pedal 4. Energy is sent from the RF generator 5 to the electrode cap 2 via the insulated wire 3, causing the electrode cap 2 to emit heat which cuts through soft tissue. As the device cuts the tissue, the heat emitted by the electrode cap 2 also cauterizes the wounds, thereby preventing bleeding.

After the tunnel for the catheter has been created, the rod 1 is removed from the body via an exit point incision. The insulated wire 3 is cut near to the rod 1 and removed from the tunnel through the entry point incision of the rod 1. Alternatively, the insulated wire 3 may be detached from the RF generator 5 and removed through the exit point incision.

An additional safety may be utilized by attaching the foot pedal 4 to a timer 11 which limits the time that the RF generator is allowed to run without releasing and re-depressing the foot pedal.

Because the conductivities of different types of tissue vary, different types of tissue may be distinguished by respective impedance values. An impedance monitor 12 may be attached to or built into the RF generator 5 for measuring the impedance of the tissue encountered by the electrode cap 2. A display screen 10 can then display the impedance values measured by the impedance monitor. (See “Measurement of Needle-Tip Bioimpedance of Facilitate Percutaneous Access of the Urinary and Biliary Systems”, the entire contents of which are incorporated herein by reference.)

In addition, an alarm 13 may be set to generate an alarm such as a flashing light, loud noise, or other warning, in response to impedance value changes exceeding a preset limit.

FIG. 3 shows a schematic view of the second embodiment of the present invention. The substantially rigid rod 1, catheter 8 and catheter holding part 9 are the same as those shown in FIG. 2. A rechargeable battery 18 is held in a casing 16, which is attachable to the substantially rigid rod 1. When the casing 16 is attached to the substantially rigid rod 1, two electrodes 15a and 15b on an outside surface of the casing connect to two electrodes 14a and 14b on an outside surface of the substantially rigid rod 1. Two wires 19a and 19b connect to the electrodes 14a and 14b and to a bipolar electrode cap 27. Because the electrode cap 27 is bipolar, no grounding pad 7 is required.

The device is activated by depressing a trigger 17 connected to the outside surface of the casing 16. When the trigger 17 is depressed, energy is transmitted from the battery 18 through the electrodes 15a and 15b, 14a and 14b and through the wires 19a and 19b to the electrode cap 2. The electrode cap 2 then emits heat which cuts through soft tissue and cauterizes the wounds, thereby preventing bleeding.

After the tunnel for the catheter has been created, the casing 16 is detached from the rod 1, which is removed from the body via an exit point incision.

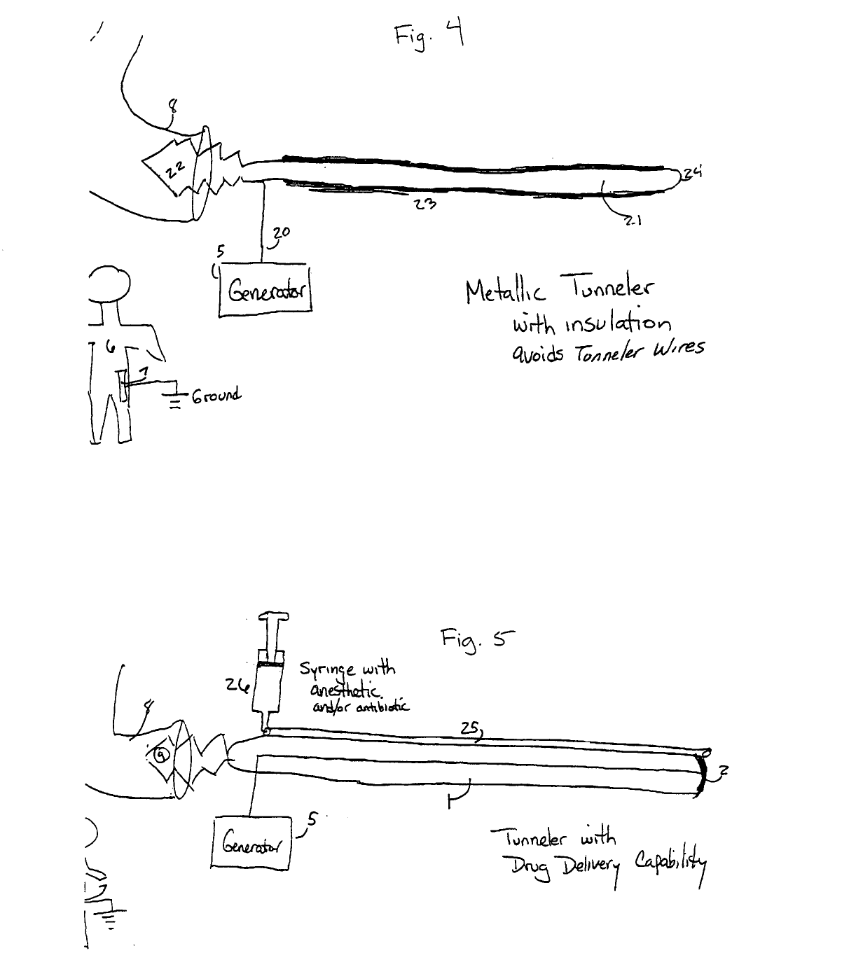

FIG. 4 shows a third embodiment of the present invention. References 5-8 identify features that are the same as those described above with respect to FIG. 1. The generator 5 is connected to a metallic rod 21 by an insulated wire 20. The metallic rod is connected to the catheter 8 by a catheter holding part 22 and is insulated except at a distal end 24 by insulation 23.

When the generator 5 is activated, energy is transmitted through the insulated wire 20 to the metallic rod 21. The energy is then emitted in the form of heat from the distal end 24 of the metallic rod 21. The heat cuts through soft tissue and cauterizes the wounds, thereby preventing bleeding.

After the tunnel for the catheter has been created, the metallic rod 21 is removed from the body via an exit point incision. The insulated wire 20 is cut near to the rod 1 and removed from the tunnel through the entry point incision of the rod 1. Alternatively, the insulated wire 20 may be detached from the RF generator 5 and removed through the exit point incision.

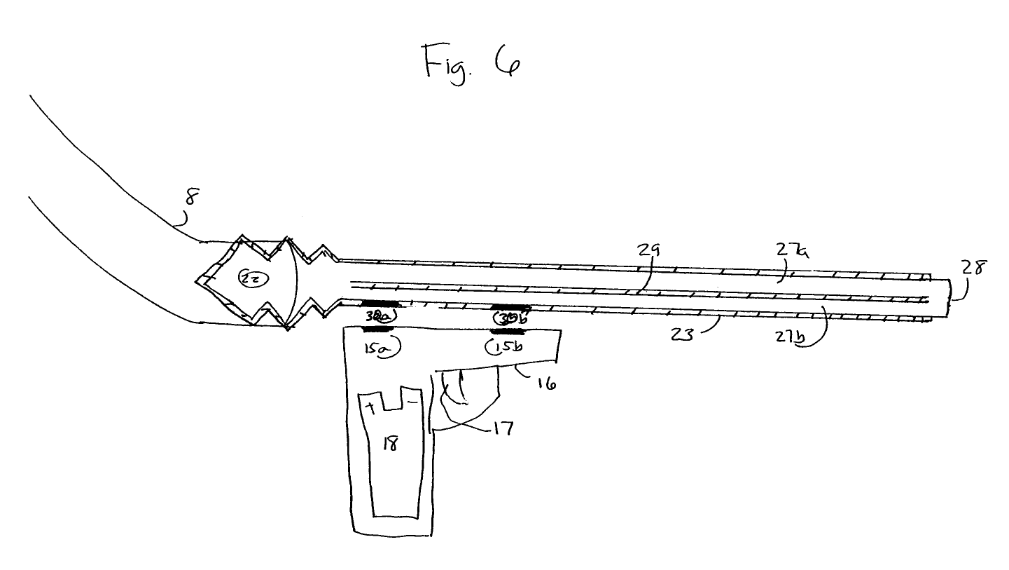

FIG. 5 shows a fifth embodiment of the present invention. References 1-9 identify features that are the same as those described above with respect to FIG. 1. A tube 25 is attached to the substantially rigid rod 1 and is connected to a syringe 26.

When the device is activated as described hereinabove, the syringe 26 is depressed to administer antibiotic or anesthetic medications through the tube 25.

FIG. 6 shows a fifth embodiment of the present invention. References 15-18 designate the electrodes, casing, trigger and battery of FIG. 3. A pair of metallic rods 27a and 27b are connected at distal and proximal ends thereof and insulated by insulation 29 between points of connection and are insulated on an exterior surface except at the distal end by insulation 23. A catheter holding part 22 connects the metallic rods to a catheter 8, and electrodes 30a and 30b connect to electrodes 15a and 15b. The device is operated in the same manner as the device of the third embodiment. When activated, energy is emitted from the distal end of the device where the metallic rods connect.

Additional advantages and modifications will readily occur to those skilled in the art. The electrode cap 2 may be replaced by an ultrasonic tip, a laser tip, or a mechanical cutting tool and the RF generator may be replaced by another power source such as a laser generator or ultrasound generator.

The impedance monitor 12, timer 11, alarm 13 and display screen 10 may comprise a device separate from the energy source. In addition, the devices of the third and fifth embodiments may be connected to another power source by a wire.

Moreover, the catheter or graft is not necessarily attached to the catheter holding part. The device may be attached to a string that is pulled through the created tunnel and which is utilized to pull the catheter through the tunnel, or the catheter or graft may be independently guided through the tunnel.

Still further, the syringe 26 and tube 25 may be used in combination with the devices of the second and third embodiments. And each of the first through third and fifth embodiments may be either monopolar or bipolar.

Therefore, the invention in its broader aspects is not limited to the specific details, representative devices, and illustrated examples shown and described herein. Accordingly, various modifications may be made without departing from the spirit or scope of the general inventive concept as defined by the appended claims and their equivalents.

1. A tunneling device for creating a path for a catheter or graft, comprising:
a substantially rigid rod;
an energy emitting part at a distal end of the rod;
an energy source connected to the energy emitting part; and
a control section for activating the energy source to transmit energy to the energy emitting part, so as to cause the energy emitting part to emit cutting and/or cauterizing energy;
wherein the substantially rigid rod is adapted to be guided through body tissue while the energy source is active so as to facilitate movement and control of the guided rod.

2. The device of claim 1, wherein the energy source comprises an RF generator and the energy emitting part comprises one of a monopolar electrode cap and a bipolar electrode cap.

3. The device of claim 2, wherein the energy emitting part comprises a monopolar electrode cap and the device further comprises a grounding pad.

4. The device of claim 1, wherein the energy emitting part comprises one of a laser tip and an ultrasonic tip, and the energy source comprises one of a laser generator and ultrasound generator, respectively.

5. The device of claim 1, wherein the energy source is connected to the energy emitting part by an insulated wire which enters the rod at or near the proximal end of the rod.

6. The device of claim 1, wherein the energy source comprises a battery housed in a casing attached to the substantially rigid rod and the control section comprises a trigger which activates the battery when the trigger is operated.

7. The device of claim 1, wherein the control section comprises a foot pedal which activates the energy source when the foot pedal is operated.

8. The device of claim 1, wherein the control section further comprises a timing section which prevents the energy source from being active for longer than a predetermined time.

9. The device of claim 1, further comprising:
a conduit; and
a syringe connected to a proximal end of the conduit;
wherein the syringe is operated to deliver medication to a patient through the conduit.

10. The device of claim 9, wherein the conduit comprises a tube attached to an external portion of the rod.

11. The device of claim 1, further comprising a catheter holding part at a proximal end of the rod for attaching a catheter or graft to the rod.

12. The device of claim 1, further comprising:
an impedance monitor for measuring impedance values of tissue contacted by the energy emitting part at the distal end of the rod;
a display screen for displaying the impedance values measured by the impedance monitor; and
an alarm generator which generates an alarm in response to a change in the impedance values measured by the impedance monitor which exceeds a predetermined parameter.

13. A tunneling device for creating a path for a catheter or graft, comprising:
a metallic rod insulated except at a distal end thereof;
an energy source connected to the rod; and
a control section for activating the energy source to transmit energy to the rod, so as to cause the distal end of the rod to emit cutting and/or cauterizing energy;
wherein the metallic rod is adapted to be guided through body tissue while the energy source is active so as to facilitate movement and control of the guided rod.

14. The device of claim 13, wherein the energy source comprises an RF generator, and the device further comprises a grounding pad.

15. The device of claim 13, wherein the energy source is connected to the rod by an insulated wire at or near the proximal end of the rod.

16. The device of claim 13, wherein the control section comprises a foot pedal which activates the energy source when the foot pedal is operated.

17. The device of claim 13, wherein the energy source comprises a battery housed in a casing attached to the metallic rod and the control section comprises a trigger which activates the battery when the trigger is operated.

18. The device of claim 13, wherein the control section further comprises a timing section which prevents the energy source from being active for longer than a predetermined time.

19. The device of claim 13, further comprising:
a conduit; and
a syringe connected to a proximal end of the conduit;
wherein the syringe is operated to deliver medication to a patient through the conduit.

20. The device of claim 19, wherein the conduit comprises a tube attached to an external portion of the rod.

21. The device of claim 13, further comprising a catheter holding part at a proximal end of the rod for attaching a catheter or graft to the rod.

22. The device of claim 13, further comprising:
an impedance monitor for measuring impedance values of tissue contacted by the energy emitting part at the distal end of the rod;
a display screen for displaying the impedance values measured by the impedance monitor; and
an alarm generator which generates an alarm in response to a change in the impedance values measured by the impedance monitor which exceeds a predetermined parameter.

23. A tunneling device for creating a path for a catheter or graft, comprising:
first and second metallic rods which are (i) attached at a proximal connection point and at a distal connection point, (ii) separated by insulating material except at the connection points, and (iii) insulated on an external surface thereof except at a distal end of said first and second rods;
a catheter holding part at a proximal end of the rod;
an energy source connected to the rod; and
a control section for activating the energy source to transmit energy to the rod, so as to cause the distal end of the rod to emit cutting and/or cauterizing energy;
wherein the connected rods are adapted to be guided through body tissue while the energy source is active so as to facilitate movement and control of the guided connected rods.
